(19) United States
(12) Patent Application Publication
(10) Pub. No.: US 2023/0034064 A1
(43) Pub. Date: Feb. 2, 2023

(54) MEDICATED PATCHING SYSTEM

(71) Applicant: Pedro Juan LOREDO, Southlake, TX (US)

(72) Inventor: Pedro Juan LOREDO, Southlake, TX (US)

(21) Appl. No.: 17/385,999

(22) Filed: Jul. 27, 2021

(51) Int. Cl. A61F 13/00; A61B 17/60; A61F 13/02

(52) U.S. Cl. CPC A61F 13/00063; A61B 17/60; A61F 13/0246; A61F 2013/00365; A61F 2013/00646

(57) ABSTRACT

A medicated patching system for positioning a plurality of medicated patches around an orthopaedic device extending through a patient's skin is provided. Each of the plurality of medicated patches includes an outer end and an opposite inner end. Each of the plurality of medicated patches also includes an upper layer and a bottom layer extending between the outer end and the inner end. A medication is positioned between the upper layer and the bottom layer. A recess extends from the inner end toward the outer end.

### TECHNICAL FIELD

The present invention is directed to medicated patches and, more particularly, to a medicated patching system for positioning a plurality of medicated patches around an orthopaedic device extending through a patient's skin.

### BACKGROUND

After orthopaedic surgery, it is common for orthopaedic devices, e.g. pins, to be inserted into a patient's bones to hold the bones in a fixed position. The orthopaedic devices are inserted through the skin and into the respective bone so that a free end of each orthopaedic device extends from the body. A fixation device may be coupled to each of the orthopaedic devices to retain the orthopaedic devices in position. An area of the patient's body surrounding each orthopaedic device may be susceptible to infections while the orthopaedic device is inserted into the body. Accordingly, it is imperative to regularly clean the area with a medication, for example an antiseptic. The disclosed embodiments provide a system and method for maintaining cleanliness of the area.

### SUMMARY

The present disclosure includes one or more of the features recited in the appended claims and/or the following features which, alone or in any combination, may comprise patentable subject matter.

According to a first aspect of the disclosed embodiments, a medicated patching system for positioning a plurality of medicated patches around an orthopaedic device extending through a patient's skin is provided. Each of the plurality of medicated patches includes an outer end and an opposite inner end. Each of the plurality of medicated patches also includes an upper layer and a bottom layer extending between the outer end and the inner end. A medication is positioned between the upper layer and the bottom layer. A recess extends from the inner end toward the outer end. A first side of a first medicated patch and a first side of a second medicated patch are configured to be positioned adjacent to and contacting each other on the patient's skin so that the recess in the first medicated patch and the recess in the second medicated patch form an opening. The orthopaedic device extends through the opening.

In some embodiments of the first aspect, the upper layer may include an impermeable layer. The bottom layer may include an adhesive layer that adheres to the patient's skin. The adhesive layer may include a plurality of pores that enable the medication to seep through the adhesive layer to the patient's skin. The medication may include an antiseptic. The opening may be sized to the orthopaedic device. The orthopaedic device may be a pin that has been inserted into a bone of the patient. The opening may be circular. The recess may be centered along a length of the inner end of each of the plurality of medicated patches. An adhesive strip may be coupled to the outer end of each of the plurality of medicated patches.

According to a second aspect of the disclosed embodiments, a medicated patch for positioning around an orthopaedic device extending through a patient's skin is provided. The patch includes a pair of outer ends and a center line equally spaced from each of the pair of outer ends. An upper layer and a bottom layer extend between the pair of outer ends. A medication is positioned between the upper layer and the bottom layer. An opening is formed at the center line and extends through the upper layer and the bottom layer. The patch is configured to be positioned on the patient's skin so that the orthopaedic device extends through the opening.

In some embodiments of the second aspect, the upper layer may include an impermeable layer. The bottom layer may include an adhesive layer that adheres to the patient's skin. The adhesive layer may include a plurality of pores that enable the medication to seep through the adhesive layer to the patient's skin. The medication may include an antiseptic. The opening may be sized to the orthopaedic device. The orthopaedic device may be a pin that has been inserted into a bone of the patient. The opening may be circular. The opening may be centered along the center line. An adhesive strip may be coupled to each outer end of the pair of outer ends.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a perspective view of an orthopaedic device coupled to a foot of a patient, wherein the orthopaedic device includes pins that are inserted into a bone of the patient;

FIG. 2 is a top view of a medicated patching system in accordance with an embodiment;

FIG. 3 is a cross-sectional view of a patch of the medicated patching system shown in FIG. 2;

FIG. 4 is a bottom view of a bottom layer of each patch of the a medicated patching system shown in FIG. 2;

FIG. 5 is a top view of a medicated patch in accordance with an embodiment; and

FIG. 6 is a bottom view of the medicated patch shown in FIG. 5.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

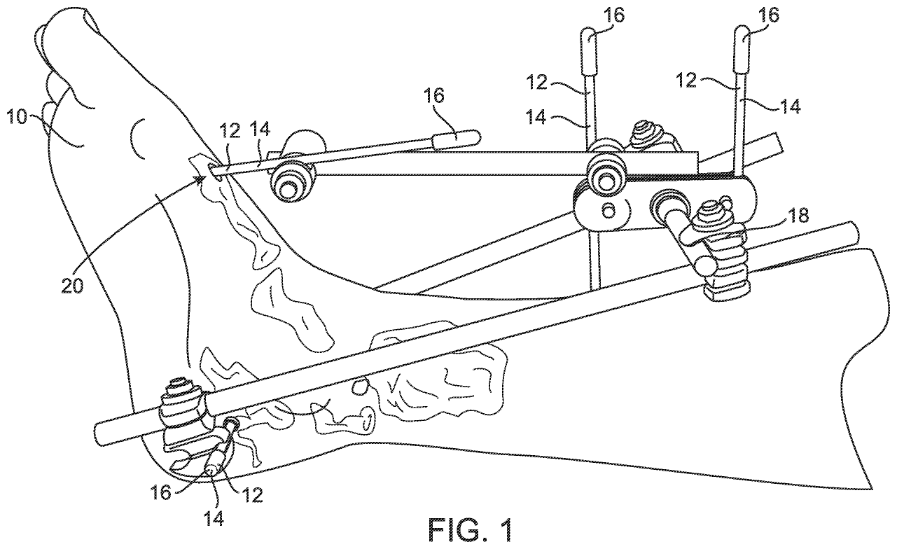

Referring to FIG. 1, a patient's foot 10 that has been operated on has orthopaedic devices 12 inserted therein. In the illustrative embodiment, the orthopaedic devices 12 include pins 14 that are inserted into bones of the patient to hold the bones in a fixed position post operation. Each of the pins 14 is inserted through the skin and the respective bone. A free end 16 of each pin 14 extends from the foot 10. A fixation device 18 is coupled to each of the pins 14 and retains the pins 14 in position. It will be appreciated that the orthopaedic devices 12 may include other devices, such as rods. Additionally, the orthopaedic devices 12 may be utilized to retain any bone in the patient's body, e.g. arm bones, hip bones, etc.

An area 20 of the patient's foot 10 surrounding each orthopaedic device 12 may be susceptible to infections while the orthopaedic device 12 is inserted into the foot 10. Accordingly, it is imperative to regularly clean the area 20 with a medication, for example an antiseptic. The disclosed embodiments provide a system and method for maintaining cleanliness of the area 20.

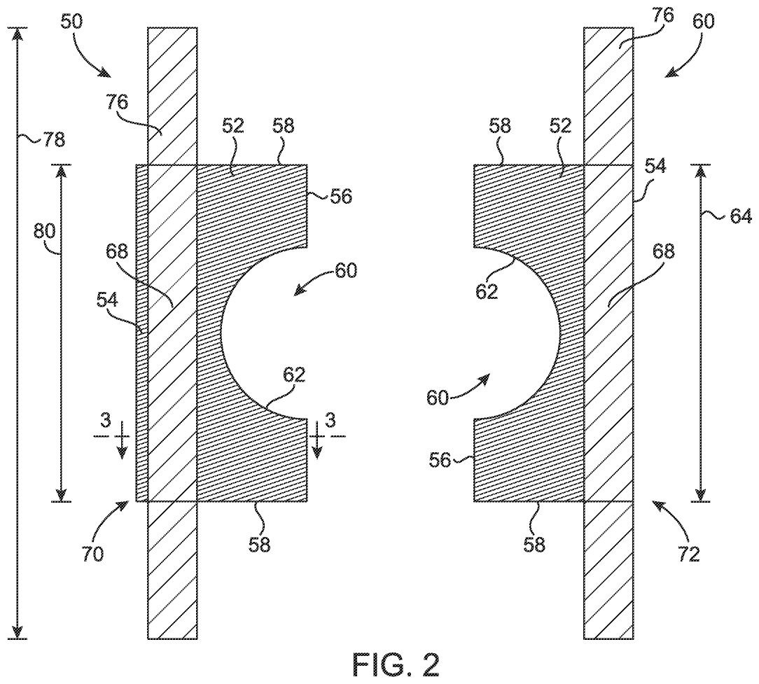

Referring now to FIG. 2, a medicated patching system 50 is configured for positioning a plurality of medicated patches 52 around the orthopaedic device 12. Each patch 52 includes an outer end 54 and an opposite inner end 56. A pair of sidewalls 58 extend between the outer end 54 and the inner end 56. A recess 60 is formed in each inner end 56. The recess 60 is defined by an edge 62 that extends inward from the inner end 56 toward the outer end 54. The recess 60 may extend any distance toward the outer end 54 so that a segment 68 of the patch 52 connects the sidewalls 58 along the outer end 54. The recess 60 is centered along a length 64 of the inner end 56. In some embodiments, the recess 60 may be offset from a center 66 of the inner end 56. The recess 60 is illustrated as a semi-circle; however, the recess 60 may take any suitable shape for positioning the patches 52 around the orthopaedic device 12.

After an orthopaedic surgery wherein the orthopaedic device 12 is inserted, the medicated patching system 50 is positioned around the orthopaedic device 12 on the patient's skin. The inner end 56 of a first medicated patch 70 and the inner end 56 of a second medicated patch 72 are configured to be positioned adjacent to and contacting each other on the patient's skin, as shown in FIG. 4. The recess 60 in the first medicated patch 70 and the recess 60 in the second medicated patch 72 form an opening 74, as illustrated in FIG. 4, when the first medicated patch 70 and the second medicated patch 72 are positioned on the patient's skin. The opening 74 is sized to the orthopaedic device 12 so that the first medicated patch 70 and the second medicated patch 72 position directly adjacent to the orthopaedic device 12. In the illustrative embodiment, the opening 74 is generally circular. In other embodiments, the shape of the opening 74 is dependent on the shape of the recesses 60 and configured based on the shape of the orthopaedic device 12. The first medicated patch 70 and the second medicated patch 72 are positioned on the patient's skin so that orthopaedic device 12 extends through the opening 74. It will be appreciated that the medicated patching system 50 may include any number of patches 52 configured to position around the orthopaedic device 12 and form the opening 74.

An adhesive strip 76 is coupled to the outer end 54 of each of the first medicated patch 70 and the second medicated patch 72. In some embodiments, the sidewalls 58 may also include an adhesive strip 76. The adhesive strip 76 extends a length 78 that is greater than a length 80 of the outer end 54. In some embodiments, the length 78 of the adhesive strip 76 is equal to the length 80 of the outer end 54. It may be contemplated that the length 78 of the adhesive strip 76 is less than the length 80 of the outer end 54. The adhesive strip 76 includes any adhesive suitable for adhering the patches 52 to the patient's skin.

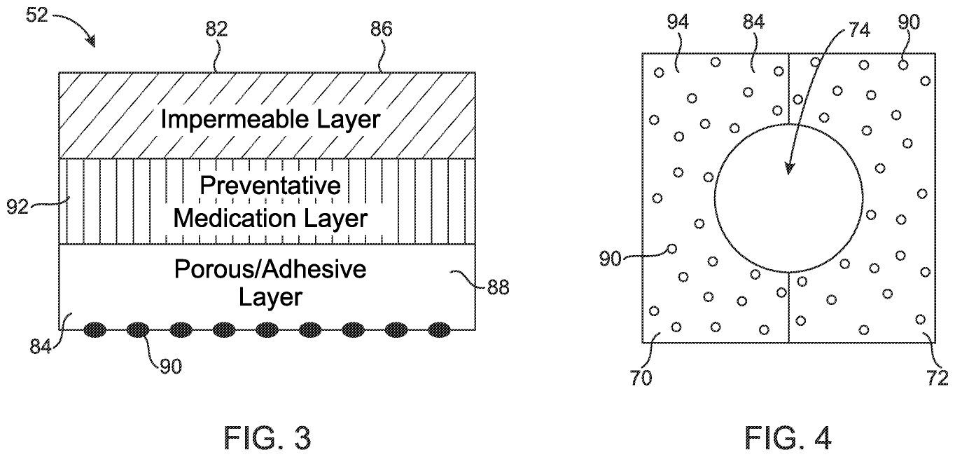

FIG. 3 illustrates a cross-sectional view of one of the patches 52 taken along ling 3-3 in FIG. 2. Each patch 52 includes an upper layer 82 and a bottom layer 84 extending between the outer end 54 and the inner end 56. The upper layer 82 includes an impermeable layer 86, e.g. plastic, that is impermeable to fluids, e.g. sweat, water, etc. The bottom layer 84 includes an adhesive layer 88 formed from an adhesive suitable for adhering the patch 52 to the patient's skin. The adhesive layer 88 includes a plurality of pores 90 that extend through the adhesive layer 88.

A medication 92 is positioned between the upper layer 82 and the bottom layer 84. The medication 92 may be an antiseptic or any other suitable medication for maintaining a sterile environment around the orthopaedic device 12. The pores 90 in the adhesive layer 88 enable the medication 92 to seep through the adhesive layer 88 to the patient's skin. As illustrated in FIG. 4, the pores 90 are distributed across a surface 94 of the adhesive layer 88. Accordingly, the medication 92 is enabled to seep through the adhesive layer 88 across the surface 94 to medicate the entire area of the patient's skin below the patch 52.

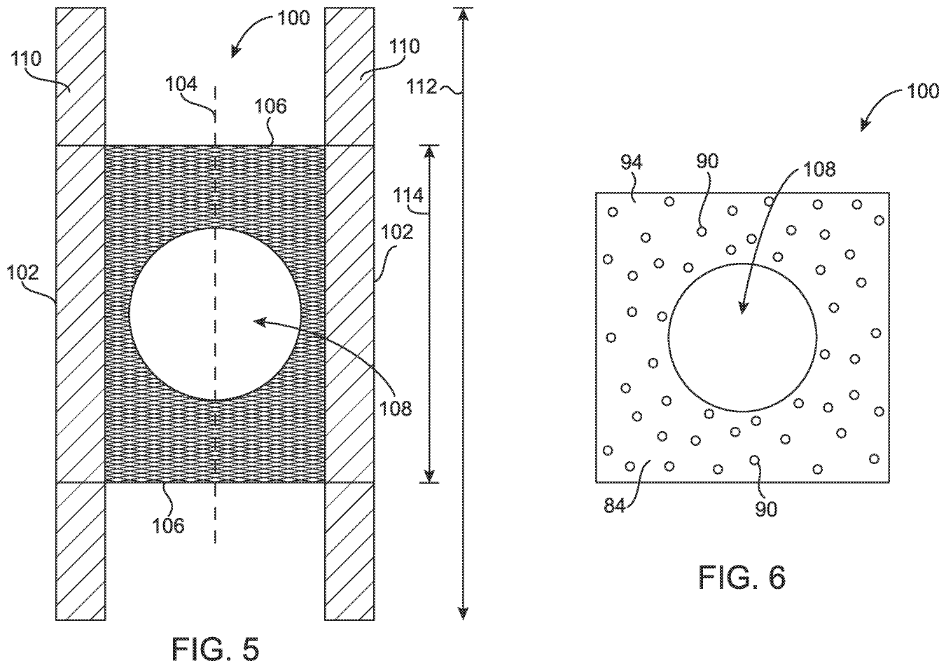

Referring now to FIG. 5, a medicated patch 100 is configured for positioning around the orthopaedic device 12. The patch 100 includes a pair of outer ends 102 and a center line 104 equally spaced from each of the pair of outer ends 102. A pair of sidewalls 106 extend between the outer ends 102. An opening 108 is formed at the center line 104 and extends through the patch 100. The patch 100 is configured to be positioned on the patient's skin so that the orthopaedic device 12 extends through the opening 108. In some embodiments, the opening 108 may be offset from the center line 104. Although the illustrative embodiment shows the opening 108 centered between the sidewalls 106, the opening 108 may be offset toward one of the sidewalls 106. The opening 108 is sized to the orthopaedic device 12 and is generally circular. In other embodiments, the shape of the opening 108 is dependent on the shape of the orthopaedic device 12.

An adhesive strip 110 is coupled to each outer end 102. In some embodiments, the sidewalls 106 may also include an adhesive strip 110. The adhesive strip 110 extends a length 112 that is greater than a length 114 of the outer end 102. In some embodiments, the length 112 of the adhesive strip 110 is equal to the length 114 of the outer end 102. It may be contemplated that the length 112 of the adhesive strip 110 is less than the length 114 of the outer end 102. The adhesive strip 110 includes any adhesive suitable for adhering the patch 100 to the patient's skin.

The patch 100 includes each of the layers described in FIG. 3. That is, the medicated patch 100 includes an upper layer 82 having an impermeable layer 86, a bottom layer 84 having an adhesive layer 88, and a medication 92, e.g. an antiseptic, positioned between the upper layer 82 and the bottom layer 84. The opening 108 extends through the upper layer 82 and the bottom layer 84. As illustrated in FIG. 4, the pores 90 formed in the bottom layer 84 are distributed across a surface 94 of the adhesive layer 88. Accordingly, the medication 92 is enabled to seep through the adhesive layer 88 across the surface 94 to medicate the entire area of the patient's skin below the patch 100.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles can have been presented, they need not be utilized in combination, and many combinations of aspects and principles are possible in light of the various embodiments provided above.

1. A medicated patching system for positioning a plurality of medicated patches around an orthopaedic device extending through a patient's skin, each of the plurality of medicated patches comprising:
an outer end and an opposite inner end,
an upper layer and a bottom layer extending between the outer end and the inner end,
a medication positioned between the upper layer and the bottom layer, and
a recess extending from the inner end toward the outer end,
wherein a first side of a first medicated patch and a first side of a second medicated patch are configured to be positioned adjacent to and contacting each other on the patient's skin so that the recess in the first medicated patch and the recess in the second medicated patch form an opening, wherein the orthopaedic device extends through the opening.

2. The medicated patching system of claim 1, wherein the upper layer includes an impermeable layer.

3. The medicated patching system of claim 2, wherein the bottom layer includes an adhesive layer that adheres to the patient's skin.

4. The medicated patching system of claim 3, wherein the adhesive layer includes a plurality of pores that enable the medication to seep through the adhesive layer to the patient's skin.

5. The medicated patching system of claim 1, wherein the medication includes an antiseptic.

6. The medicated patching system of claim 1, wherein the opening is sized to the orthopaedic device.

7. The medicated patching system of claim 1, wherein the orthopaedic device is a pin that has been inserted into a bone of the patient.

8. The medicated patching system of claim 1, wherein the opening is circular.

9. The medicated patching system of claim 1, wherein the recess is centered along a length of the inner end of each of the plurality of medicated patches.

10. The medicated patching system of claim 1, further comprising an adhesive strip coupled to the outer end of each of the plurality of medicated patches.

11. A medicated patch for positioning around an orthopaedic device extending through a patient's skin, the patch comprising:
a pair of outer ends and a center line equally spaced from each of the pair of outer ends,
an upper layer and a bottom layer extending between the pair of outer ends,
a medication positioned between the upper layer and the bottom layer, and
an opening formed at the center line and extending through the upper layer and the bottom layer,
wherein the patch is configured to be positioned on the patient's skin so that the orthopaedic device extends through the opening.

12. The medicated patch of claim 11, wherein the upper layer includes an impermeable layer.

13. The medicated patch of claim 12, wherein the bottom layer includes an adhesive layer that adheres to the patient's skin.

14. The medicated patch of claim 13, wherein the adhesive layer includes a plurality of pores that enable the medication to seep through the adhesive layer to the patient's skin.

15. The medicated patch of claim 11, wherein the medication includes an antiseptic.

16. The medicated patch of claim 11, wherein the opening is sized to the orthopaedic device.

17. The medicated patch of claim 11, wherein the orthopaedic device is a pin that has been inserted into a bone of the patient.

18. The medicated patch of claim 11, wherein the opening is circular.

19. The medicated patch of claim 11, wherein the opening is centered along the center line.

20. The medicated patch of claim 11, further comprising an adhesive strip coupled to each outer end of the pair of outer ends.
